(19) United States
(12) Patent Application Publication
(10) Pub. No.: US 2019/0046570 A1
(43) Pub. Date: Feb. 14, 2019

(54) Compositions and Methods for Recombinant CXADR Expression

(71) Applicants: NANT HOLDINGS IP, LLC, Culver City, CA (US); NANTCELL, INC., Culver City, CA (US)

(72) Inventors: Patrick Soon-Shiong, Culver City, CA (US); Shahrooz Rabizadeh, Culver City, CA (US); Kayvan Niazi, Culver City, CA (US)

(21) Appl. No.: 16/073,947

(22) PCT Filed: Feb. 3, 2017
(86) PCT No.: PCT/US17/16543

§ 371 Date: Jul. 30, 2018

Related U.S. Application Data
(60) Provisional application No. 62/291,999, filed on Feb. 5, 2016.

(51) Int. Cl. A61K 35/17; C12N 15/85; C12N 15/11; A61P 35/00; C07K 14/705; C12N 5/0781; C12N 5/0783; C12N 5/0784; A61K 48/00

(52) U.S. Cl. CPC A61K 35/17; C12N 15/85; C12N 15/111; A61P 35/00; C07K 14/70503; C12N 5/0635; A61K 48/0058; C12N 5/0639; C12N 5/0646; A61K 48/0075; A61K 48/0083; A61K 48/0066; C12N 5/0636

(57) ABSTRACT

Recombinant expression of CXADR in a cell, and especially an immune competent cell is employed to enable or improve gene delivery to the cell by an adenovirus. In particularly preferred aspects, the immune competent cell is a an NK cell, a T-cell, a B-cell, a macrophage, or a dendritic cell, and the gene delivery comprises a recombinant nucleic acid encoding a disease-specific antigen, such as a patient specific neoepitope or a tumor associated antigen.

This application claims priority to US provisional application with the Ser. No. 62/291,999, filed Feb. 5, 2016.

### FIELD OF THE INVENTION

The field of the invention is compositions and methods of genetic modification of cells, and especially modifications that render cells sensitive to viral infection.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Adenoviruses are well-characterized double stranded DNA viruses and known for their ability to cause respiratory infection in man. More recently, the adenoviral genome was modified to generate compositions that enable the production of adenovirus particles that contain various transgenes for delivery to many cell types of interest. Adenovirus type 5 represents one of the best studied platforms in this regard, with numerous kits available in the commercial space to produce user-determined viruses (e.g., Vector BioLabs, USA, Malvern, Pa. 19355; or Thermo Fisher Scientific, USA, Waltham, Mass. 02451). Adenovirus type 5 produced in this manner have been used in cell culture, animal, and even clinical trials, further supporting the familiarity of scientific and clinical practitioners with this system. Entry of the virus into the cell is thought to be mediated via the Coxsackie and Adenovirus receptor (CXADR).

Cells or tissues that fail to produce CXADR or produce insufficient quantities of CXADR for viral entry have limited the use of the Adenovirus type 5 technology in such cells, and so prevent transduction of many clinically relevant cells and tissues, including stem cells and immune cells. CXADR (Swiss-Prot Accession Number: P78310) is a type I membrane receptor and a member of the immunoglobulin superfamily (Science (1997) 275; 1320-1323). CXADR has an extracellular domain that is typically larger than 200 amino acids in size and is believed to be a component of the epithelial apical junction complex essential for the tight junction integrity (J Biol Chem (1999) 274; 10219-10226). CXADR recruits intracellular PDZ domain-containing protein LNX (Ligand-of-Numb Protein-X) to intercellular contact sites (J Biological Sci (2003) 278; 7439-7444). CXADR may also function as a homophilic cell adhesion molecule (Molecular Brain Research (2000) 77; 19-28) and has been observed in transepithelial migration of PMN through adhesive interactions with JAML located in the plasma membrane of PMN (Mol Biol Cell (2005) 16; 2694-703). CXADR knockout mice exhibited embryonic lethal phenotype associated with cardiac defects (Genesis (2005) 42; 77-85). Based on these multiple functions and involvements, the primary physiological role of CXADR is unlikely a viral entry receptor.

Over-expression of CXADR has been observed in osteosarcomas and malignant thyroid tumors (Cancer Sci (2003) 94; 70-75; Thyroid (2005) 15; 977-87), and CXADR was also over-expressed in breast, kidney, and lung cancer cell lines, and in colon tumor tissues as described in US 2014/0193419. Notably, a CXADR antisense plasmid vector abrogated xenografts mediated by high expressing lung cancer cells and inhibited soft agar colony formation (Cancer Res (2004) 64; 6377-80). CXADR expression is enhanced after transition from preneoplastic precursor lesions to neoplastic mammary cancer outgrowth in a syngenic mouse tumor model (Clin Cancer Res (2005) 11; 4316-20). In a 3D tissue culture model of breast cancer cells, disruption of polarity and integrity, as in malignant transformation, can lead to up-regulation of CXADR (Proc. Natl. Acad. Sci. (2003) 100, 1943-1948). CXADR over-expression in ovarian and cervical cancer cell lines enhanced cell survival by protecting against apoptosis (Clin Cancer Res (2005) 11; 4316-20). Expression of CXADR in gastrointestinal cancers correlated with tumor differentiation (Cancer Gene Ther (2006) Epub). Loss of CXADR expression associated with advanced bladder cancer (Urology (2005) 66; 441-6). Over-expression of CXADR in an ovarian cancer cell line inhibited cell migration (Exp Cell Res (2004) 298; 624-31). Expression of CXADR decreased in primary prostate cancer but is highly expressed upon metastasis (Cancer Res (2002) 62; 3812-8).

In known uses of CXADR, as disclosed in US 2015/0140018 an antibody capable of binding to an epitope present at positions 181 to 230 of human CXADR was reported to have anti-cancer activity against prostate cancer cells, pancreatic cancer cells, and colorectal cancer cells. Furthermore, the '018 application disclosed that the antibody had ADCC and CDC activity. In addition, as described in US 2008/0124360, adenoviral vectors have been constructed to give rise to modified or heterologous fiber proteins suitable for targeting dendritic cells to so more specifically deliver antigens to dendritic cells for processing and presentation to T cells. To that end, viral particles were de-targeted from binding to certain native receptors (e.g., coxsackie-adenovirus receptor for Ad5 and Ad2), and re-targeted to receptors expressed on dendritic cells. While such re-targeting is at least conceptually beneficial with respect to redirection of infection, new difficulties arise. Among other things, viral propagation in established adenovirus host cells is no longer a choice due to the loss binding to the CXADR receptor required for infection of the host cells.

Notably, however, expression or over-expression of CXADR in therapeutic cells does not appear to be have been used in cancer therapy or even in a supporting role of treatment of a patient diagnosed with cancer. Therefore, there is still a need for compositions and methods using CXADR in such settings.

### SUMMARY OF THE INVENTION

The inventive subject matter is directed to compositions and methods of treatment of cancer in which recombinant expression of CXADR in an immune competent cell is used to increase susceptibility of the cell to viral transfection, and particularly transfection with a recombinant adenovirus. The modified cells are contemplated to provide therapeutic function in a direct (e.g., a dendritic or NK cell infected with a recombinant adenovirus that encodes patient and tumor specific neoepitopes) or indirect (e.g., NK cell infected with a recombinant adenovirus that encodes a co-stimulatory molecule or checkpoint inhibitor) manner.

In one aspect of the inventive subject matter, the inventors contemplate a method of modifying an immune competent cell that comprises a step of introducing into the immune competent cell a recombinant nucleic acid encoding CXADR to produce a modified immune competent cell. In a further step, the modified immune competent cell is cultivated in a first medium under conditions to express the CXADR.

Most typically, the immune competent cell is an NK cell (e.g., immortalized or an NK92 cell, or a genetically engineered NK92 cell), a T-cell (e.g., CD8+), a B-cell, a macrophage, or a dendritic cell. For example, the NK cell may be genetically engineered to have a reduced or abolished expression of at least one killer cell immunoglobulin-like receptor (KIR), may be genetically engineered to express a high-affinity Fcγ receptor, or may be genetically engineered to express a chimeric T-cell receptor. With respect to suitable regulatory elements, it is contemplated that the CXADR gene may be under the control of a constitutively active promoter, a NK cell specific promoter, or a hypoxia inducible promoter. It is furthermore contemplated that suitable NK cells may be naturally permissive to a particular adenovirus, or that the NK cell is modified or selected to increase or exhibit permissivity with respect to one or more specific adenovirus.

As will be further appreciated, contemplated methods may further comprise a step of infecting the modified immune competent cell with a recombinant adenovirus (preferably having an E2b deletion) that will include a recombinant nucleic acid that encodes a (typically patient and tumor specific) neoepitope, a co-stimulatory molecule, a cytokine, and/or a checkpoint inhibitor. With respect to suitable adenoviruses, it should be noted that preferred adenoviruses will be readily able to infect the immune competent cell and/or permit expression of one or more gene transferred into the infected cell. It is further noted that contemplated methods may further comprise a step of administering the modified immune competent cell to a patient, and that the step of infecting is performed in vivo after administration of the modified immune competent cell to the patient. Alternatively, contemplated methods may also comprise a step of administering the modified immune competent cell to a patient, wherein the step of infecting is performed in vitro before administration of the modified immune competent cell to the patient.

Where desired, the modified immune competent cell is autologous to a patient to which the modified immune competent cell is administered, and/or may be propagated in the first medium to a desired quantity, which is then replaced with a second medium suitable for administration of the modified immune competent cell.

Therefore, the inventors also contemplate a genetically modified immune competent cell that includes a recombinant nucleic acid encoding CXADR (e.g., isoform 1) operably coupled to a regulatory sequence for expression of the CXADR in the immune competent host cell. As noted above, suitable immune competent cells include NK cells, T-cells, B-cells, macrophages, and dendritic cells. However, in further contemplated aspects, alternate cells need not necessarily be immune competent cells, and suitable other cells expressly include CHO cells, HEK-293 cells, mouse myeloma lymphoblastoid cells, BHK cells, Sf9 cells, etc.

Where the genetically modified immune competent cell is an NK cell, such NK cells may be genetically modified NK cell, an NK92 cell, or a NK92 derivative. For example, the cell may be genetically modified to express a high-affinity Fcγ receptor (which may be coupled to an antibody that has binding specificity against a tumor associated antigen, a tumor specific antigen, or a cancer neoepitope), or may be genetically modified to express a chimeric T-cell receptor (e.g., comprising an scFv portion). Preferred chimeric T-cell receptor will typically have an ectodomain with binding specificity against a tumor associated antigen, a tumor specific antigen, or a cancer neoepitope.

The recombinant nucleic acid in genetically modified immune competent cells may be incorporated into the genome of the host cell, or be present as an extrachromosomal DNA or RNA. Most typically, but not necessarily, the regulatory sequence may comprise an NK cell specific promoter or a hypoxia inducible promoter.

Viewed from a different perspective, the inventors also contemplate a method of conditioning a patient for immunotherapy of a cancer. Preferred methods include a step of administering to the patient an immune competent cell (e.g., NK cell, T-cell, B-cell, macrophage, or dendritic cell) that is genetically modified to express CXADR.

Moreover, such methods may include a further step of infecting the immune competent cell with a recombinant adenovirus that comprises a nucleic acid that encodes at least one of a neoepitope, a co-stimulatory molecule, a cytokine, and a checkpoint inhibitor, or include a further step of administering to the patient a recombinant adenovirus that comprises a nucleic acid that encodes at least one of a neoepitope, a co-stimulatory molecule, a cytokine, and a checkpoint inhibitor. Preferably, the recombinant adenovirus will have a deleted or non-functional E2b gene.

In still further aspects of the inventive subject matter, a method of treating a patient diagnosed with cancer is contemplated that includes a step of administering to the patient a genetically modified immune competent cell that comprises a recombinant nucleic acid that encodes CXADR and in which the nucleic acid that encodes CXADR is operably coupled to a regulatory sequence for expression of the CXADR in the immune competent host cell. In another step, a recombinant adenovirus is administered to the patient that comprises a nucleic acid that encodes a neoepitope, a co-stimulatory molecule, a cytokine, and/or a checkpoint inhibitor. Most typically, the recombinant adenovirus is administered upon expression of the CXADR in the genetically modified immune competent cell in the patient.

Preferred immune competent cells include NK cells, T-cells, B-cells, macrophages, and dendritic cells, and it is generally preferred that the recombinant adenovirus has a deleted or non-functional E2b gene, and/or that the genetically modified immune competent cell is an autologous cell of the patient.

In still contemplated aspects a method of treating a patient diagnosed with cancer is contemplated. Such method will typically include a step of infecting a genetically modified immune competent cell with a recombinant adenovirus, wherein the genetically modified immune competent cell comprises a recombinant nucleic acid that encodes CXADR and in which the nucleic acid that encodes CXADR is operably coupled to a regulatory sequence for expression of the CXADR in the immune competent host cell, and wherein the recombinant adenovirus comprises a nucleic acid that encodes at least one of a neoepitope, a co-stimulatory molecule, a cytokine, and a checkpoint inhibitor. In another step, the infected immune competent cell is administered to the patient.

Thus, use of a genetically modified immune competent cell in the treatment of cancer is also contemplated, wherein the genetically modified immune competent cell is a genetically modified immune competent cell as presented herein. It is still further noted that the genetically modified immune competent cell may be a cell that is infected with a recombinant adenovirus that comprises a nucleic acid that encodes at least one of a neoepitope, a co-stimulatory molecule, a cytokine, and a checkpoint inhibitor.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an exemplary schematic of a CXADR sequence cassette suitable for use in conjunction with the teachings herein.

### DETAILED DESCRIPTION

Recombinant adenovirus (AdV) type 5 provides a well-characterized and commonly used viral gene delivery platform in permissive cell types. However, the transformation potential of a given cell type for AdV type 5 is predominantly dependent on the expression of CXADR or sufficient quantities thereof. Unfortunately, the expression of this receptor is notoriously absent in many cancer types, stem cells, or adoptively transferred immune cells (such as cytotoxic T cells or NK cells).

The inventors have now discovered that cells can be transfected with a nucleic acid construct to facilitate expression of the CXADR gene to a cell of interest, and to so impart sensitivity to AdV type 5 transfection in vitro and in vivo. Most preferably, cells suitable for recombinant expression of CXADR include immune competent cells, such as NK cells, T-cells (CD8+, CD4+, etc.), B-cells, macrophages, and certain dendritic cell populations. By expression of the CXADR it is contemplated that these genetically engineered cells will now be susceptible to infection with a genetically modified adenovirus to deliver a recombinant nucleic acid to the so infected cells. Most preferably, the delivery is performed using an adenoviral construct that has reduced or abolished immunogenicity, and particulary contemplated adenoviruses include those in which the E2b gene is non-functional or deleted (see e.g., Journal Of Virology, February 1998, p. 926-933).

In this context, it should be appreciated that transfection of a host cell (e g, immune competent cell such as an NK cell or protein production cell) with the CXADR gene need not necessarily result in a generally permissive cell that allows infection with a large variety of viruses. Indeed, it should be appreciated that (e.g., depending on the isotype of CXADR used) infection of the transfected cell may be restricted to specific subset of viruses and even subsets of adenoviruses. For example, while some transfected immune competent cells (e.g., NK92 cells) may be readily susceptible to infection with primate (e.g., gorilla) derived adenoviruses, they may be less susceptible to transfection with human or modified adenoviruses. On the other hand, some modified adenoviruses (e.g., NK92 derivatives) may readily infect immune competent cells due to their modification (e.g., where certain early genes were removed to remove innate immunogenicity).

Moreover, and especially in situations where the transfected cells are less permissive to viral infection, it is contemplated that cells may be adapted to/selected for permissivity. Such selection may be clonal expansion where the cells are immortalized, or cells may first be immortalized and then transfected and selected for permissivity. Alternatively, transfected permissive cells may also be analyzed for one or more traits establishing permissivity, and these traits may then be imparted to further cells for improvement in permissivity.

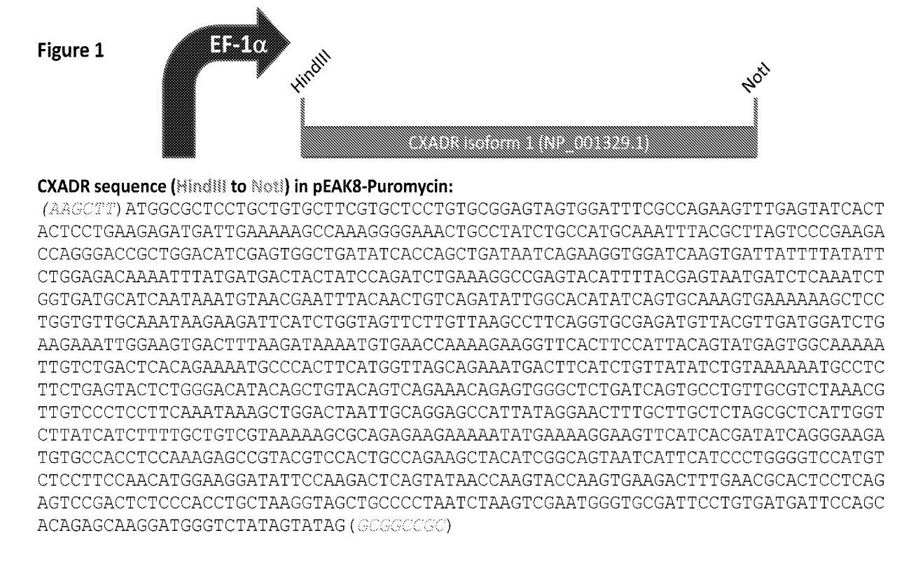

In one exemplary aspect of the inventive subject matter, a cDNA encoding CXADR was amplified from a HEK-293T total cDNA preparation and subsequently cloned into the peak8-puromycin plasmid as is exemplarily shown in FIG. 1. Gene expression was driven from EF-1α, the human elongation factor 1 promoter. The so prepared recombinant sequence was verified by DNA sequencing and aligned perfectly with the known sequence for human CXADR isoform 1 in a reference data set (NP_001329.1). The expression plasmid was then transfected into NK92 cells using standard transfection protocols well known in the art. Selection of transfected cells for preparation of a cell stock was performed using puromycin. Such transformed cells are especially advantageous as human NK cell lines are generally known to be difficult to transduce with adenoviruses, and especially AdV type 5.

Of course, it should be appreciated that the inventive subject matter is not limited to the specific expression vector noted above, and that indeed all manners of expression from a recombinant nucleic acid in a cell are deemed suitable for use herein. In general, suitable CXADR encoding nucleic acid sequences can be cloned into a number of types of vectors. For example, the CXADR nucleic acid can be cloned into a circular vector such as a plasmid, a phagemid, a phage derivative, an animal virus, and a cosmid. Vectors of particular interest include expression vectors, replication vectors, probe generation vectors, and sequencing vectors. Further, the expression vector may be provided to a cell in the form of a viral vector. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include various retroviruses, adenoviruses, adeno-associated viruses, herpes viruses, and lentiviruses. In general, suitable vectors will contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of well known viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. In some embodiments, adenovirus vectors or lentivirus vectors are used. Of course, it should be appreciated that the inventive subject matter is not limited to a specific vector, and that indeed all manners of expression from a recombinant nucleic acid in a cell are deemed suitable for use herein, including expression form a construct other than a vector. For example, where transient expression is desired, the recombinant nucleic acid may be delivered as an RNA or as extrachromosomal DNA without eukaryotic replication sequence. On the other hand, where permanent expression is desired, the nucleic acid may be delivered for integration into the cell's genome, or the cell may be subject to genome editing (e.g., using CRISPR/Cas9 technology) to so install an expression cassette into the genome.

Likewise, it should be appreciated that the transcription and translation control may vary considerably, and expression may be driven from a constitutively active promoter, from an inducible promoter using corresponding inducing agents, or from a promoter that is activated under selected tissue or culture conditions. As is known in the art, various promoter elements (e.g., initiation factor binding sites, polymerase binding sites, enhancers, etc.) regulate the frequency of transcriptional initiation. Typically, these are located in the region 30-110 bp upstream of the start site, although a number of promoters have been shown to contain functional elements downstream of the start site as well. The spacing between promoter elements frequently is often flexible, so that promoter function is preserved when elements are inverted or moved relative to one another. For example, in the thymidine kinase (tk) promoter, the spacing between promoter elements can be increased to 50 bp apart before activity begins to decline.

Depending on the promoter, it should also be appreciated that individual elements can function either cooperatively or independently to activate transcription. Exemplary promoters include the CMV IE gene, EF-1a, ubiquitin C, or phosphoglycerokinase (PGK) promoters. In further contemplated aspects, the promoter is a PGK promoter, or a promoter that is capable of expressing a CXADR transgene in a mammalian T cell, such as the EF-1a promoter. The native EF-1 a promoter drives expression of the alpha subunit of the elongation factor-1 complex, which is responsible for the enzymatic delivery of aminoacyl tRNAs to the ribosome. The EF-1a promoter has been extensively used in mammalian expression plasmids and has been shown to be effective in driving expression from transgenes cloned into various viral vectors (see, e.g., Mol. Ther. (2009), 17(8): 1453-1464).

Further examples of suitable promoters include the immediate early cytomegalovirus (CMV) promoter. This promoter sequence is a strong constitutive promoter sequence capable of driving high levels of expression of any polynucleotide sequence operatively linked thereto. However, other constitutive promoter sequences may also be used, including the simian virus 40 (SV40) early promoter, the mouse mammary tumor virus (MMTV), the human immunodeficiency virus (HIV) long terminal repeat (LTR) promoter, the MoMuLV promoter, the avian leukemia virus promoter, the Epstein-Barr virus immediate early promoter, the Rous sarcoma virus promoter, as well as human gene promoters such as the actin promoter, the myosin promoter, the hemoglobin promoter, and the creatine kinase promoter. Furthermore, it should be appreciated that the inventive subject matter is not limited to constitutive promoters, but that inducible promoters are also expressly contemplated herein. The use of an inducible promoter advantageously provides a molecular switch that is capable of turning on expression of a polynucleotide sequence (which is operatively linked to the inducible promoter) when such expression is desired, and turning off the expression when expression is not desired. Examples of inducible promoters include the metallothionine promoter, the glucocorticoid promoter, the progesterone promoter, and the tetracycline promoter.

Where it is desired that expression of the recombinant CXADR is limited to NK cell specific expression, expression constructs are contemplated that comprise a promoter that is specific for genes that are preferentially (expression in less than 10, or less than 6, but more than 2 tissues as listed in The Human Protein Atlas; URL: www.proteinatlas.org) or even exclusively expressed (expression in less than 3, but more than 0 tissues as listed in The Human Protein Atlas) in NK cells. Similarly, promoters with tissue or cell specific expression for other immune competent or antigen presenting cells (e.g., CD8+ T cells, CD4+ T cells, macrophages, dendritic cells) are also expressly deemed suitable for use herein.

For example, where expression of the CXADR is preferred in or limited to NK cell specific expression, a mammalian NK cell receptor promoter may be operably linked to the CXADR sequence. Suitable promoters may be (derived) from the NKp30 promoter (see, e.g., J Exp Med (1999), 190:1505-1516), from the NKp44 promoter (see, e.g., J Exp Med (1999), 189:787-796), and from the NKp46 promoter (see, e.g. J. Exp. Med (1997), 186:1129-1136; J Exp Med (1998), 188(5):953-60; or Nature (2001), 409:1055-1060). While human sequences are preferred, alternative sources, and especially mammalian sources are also deemed suitable herein. Sequences, genetic and motif information, homology, and other relevant information, including information about homologs in other organisms for such genes are readily available (see e.g., human NKp30 Gene ID: 259197; NKp44 Gene ID: 9436; NKp46 Gene ID: 9437). Moreover, additional elements, for example, enhancers located downstream of the coding sequence of the gene, can be used in conjunction with the teachings presented herein.

In still further contemplated aspects, contemplated promoters may also be sensitive to one or more environmental conditions to drive the transcription of the CXADR gene. For example, expression may be driven under the control of a temperature sensitive promoter (see e.g., BMC Biotechnol. 2011; 12; 11:51) or under the control of a hypoxia and metal sensitive promoter (see e.g., Gene Ther. 2006; 13(10):857-68). Such control may be particularly advantageous where the cells are used in cancer therapy as many tumors present a hypoxic microenvironment.

Regardless of the particular type and nature of the promoter it is contemplated that the promoter will be operably linked to the CXADR sequence to so drive expression in the host cell (i.e., cell transformed with the vector or other expression construct). As will be readily appreciated, the recombinant nucleic acid construct may include various additional elements, including transcription termination elements, intronic sequences, and/or polyadenylation signals. Construction of expression constructs can be accomplished using any suitable genetic engineering techniques, including, inter alia, restriction endonuclease digestion, ligation, transformation, plasmid purification, and DNA sequencing. Such techniques are well known in the art and are described elsewhere (see e.g., in Sambrook et al., Molecular Cloning: A Laboratory Manual (Cold Spring Harbor Laboratory, N.Y., (1989)).

In still further contemplated aspects of the inventive subject matter, it should be appreciated that the expression of the CXADR is not limited to isoforms 1 as exemplarily set out above. Indeed, suitable CXADR proteins include all proteins that act as a coxsackievirus and adenovirus receptor and as such mediates entry of a coxsackievirus and/or adenovirus (and especially adenovirus type 5) into a cell. For example, one suitable human CXADR isoform 1 protein sequence is described in NP_001329 (which is encoded by corresponding nucleic acid sequence NM_001338).

However, numerous alternative isoforms and precursors for human CXADR proteins are also deemed appropriate and include Isoform 4 precursor (e.g., NP_001193994.1), Isoform 2 precursor (e.g., NP_001193992.1), Isoform 3 precursor (e.g., NP_001193993.1), Isoform X1 (e.g., XP_011527778.1), Isoform X2 (e.g., XP_011527779.1), Isoform X3 (e.g., XP_011527780.1), Isoform X4 (e.g., XP_011527781.1), Isoform CRA_b (e.g., EAX10031.1), Isoform CRA_d (e.g., EAX10033.1), etc. Likewise, the CXADR need not be limited to the human protein, but may also be a murine CXADR protein (e.g., NP_001020363.1), a rat CXADR protein (e.g., NP_446022.1), or a bovine CAXDR protein (e.g., NP_776723.1). Of course, and with respect to the nucleic acid encoding the CXADR protein, all corresponding nucleic acid sequences are deemed appropriate. Most preferably, the nucleic acid sequences will be optimized for human codon usage and/or increased expression.

Moreover, it should also be appreciated that all protein sequences and corresponding nucleic acid sequences contemplated herein may vary to some degree from any sequences as described above, and variations may be due to rational-based base changes (e.g., to introduce a restriction site, to codon optimize, to add functionalities for later modifications, etc.) or due to inadvertent mutations. Therefore, the expressed CXADR protein will be is at least about 30%, 35%, 40%, 45% or 50%, preferably at least about 55%, 60%, 65% or 70%, and more preferably at least about 75%, 80%, 85%, 90%, 91%, 92%, 93% or 94% and most preferably at least about 95%, 97%, 98%, 99% or more homologous to the sequences as described above.

It will be appreciated that the expression vectors described herein are useful both for producing recombinant non-mammalian NK cells and human NK cells, which may be freshly isolated, cultured from precursor or stem cells, or from existing cultures (which may be genetically modified). There are numerous methods of introducing and expressing genes into a cell known in the art. In the context of an expression vector, the vector can be readily introduced into a NK cell or other host cell (and especially immune competent cells capable of presenting an antigen via MHC complexes) by any method known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, etc. Methods for producing cells comprising vectors and/or exogenous nucleic acids are well-known in the art. See, for example, Sambrook et al., 2012, Molecular Cloning: A Laboratory Manual, volumes 1-4, Cold Spring Harbor Press, NY). A suitable method for the introduction of a polynucleotide into a host cell is calcium phosphate transfection or lipofection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, etc (see, for example, U.S. Pat. Nos. 5,350,674 and 5,585,362).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system. In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Also contemplated are lipofectamine-nucleic acid complexes. Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

With respect to cells for transfection, it is contemplated that all cells are deemed suitable for use herein, and especially cells that have no or only a relatively low expression of endogenous CXADR. For example, suitable cells include immune competent cells, such as NK cells, T-cells (CD8+, CD4+, etc.), B-cells, macrophages, and dendritic cells, but also cells from kidney, placenta, thymus, and spleen, and certain tumor cells (advanced bladder cancer, primary prostate cancer, etc.) that have low levels of CXADR expression. In general, and viewed from another perspective, it is contemplated that all cells are suitable for transfection that are desired to be transfected with an adenoviral vector at a later time (i.e., after expressing the recombinant CXADR). However, immune competent cells, and especially NK cells and modified NK cells are particularly preferred as expression of CXADR in such cells allows transfection in vivo with a recombinant nucleic acid (via adenoviral delivery) that can deliver one or more antigens (and particularly neoantigens, tumor associated antigens, or chimeric molecules comprising such antigens) to the immune system in a host.

NK cells can be readily identified by virtue of certain characteristics and biological properties, such as the expression of specific surface antigens including CD56 and/or CD16 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Of course, it should be noted that suitable host cells, and particularly NK cells are either obtained from the patient diagnosed with the tumor, or are obtained from an already established cell line as further detailed below.

For example, in one particularly preferred aspect of the inventive subject matter, the NK cell is a NK-92 derivative and is preferably genetically modified to have a reduced or abolished expression of at least one killer cell immunoglobulin-like receptor (KIR), which will render such cells constitutively activated (via lack of or reduced inhibition). Therefore, suitable modified cells may have one or more modified killer cell immunoglobulin-like receptors that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated KIR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and KIR3DS1. Such modified cells may be prepared using protocols well known in the art. Alternatively, such cells may also be commercially obtained from NantKwest (see URL www.nantkwest.com) as aNK cells (‘activated natural killer cells).

In another example, the genetically engineered NK cell may also be an NK-92 derivative that is modified to express the high-affinity Fcγ receptor (CD16). Sequences for high-affinity variants of the Fcγ receptor are well known in the art, and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells (‘high-affinity natural killer cells). Such cells may then be further modified to express the CXCL12 or portion thereof or to have reduced or abolished expression of CXCR4 as also further discussed below.

In yet a further aspect of the inventive subject matter, the genetically engineered NK cell may also be genetically engineered to express a chimeric T-cell receptor. In especially preferred aspects, the chimeric T-cell receptor will have a scFv portion or other ectodomain with binding specificity against a tumor associated antigen, a tumor specific antigen, and a cancer neoepitope. As noted before, there are numerous manners of genetically engineering an NK cell to express such chimeric T-cell receptor, and all manners are deemed suitable for use herein. Alternatively, such cells may also be commercially obtained from NantKwest as taNK cells (‘target-activated natural killer cells’). Such cells may then be further modified to express the CXCL12 or portion thereof or to have reduced or abolished expression of CXCR4 as discussed below.

Where the cells are engineered to have affinity towards a cancer associated antigen or antibody with specificity towards a cancer associated antigen, it is contemplated that all known cancer associated antigens are considered appropriate for use. For example, cancer associated antigens include CEA, MUC-1, CYPB1, etc. Likewise, where the cells are engineered to have affinity towards a cancer specific antigen or antibody with specificity towards a cancer specific antigen, it is contemplated that all known cancer specific antigens are considered appropriate for use. For example, cancer specific antigens include PSA, Her-2, PSA, brachyury, etc.

In addition, it is contemplated that the NK or other host cells (e.g., immune competent cells) may be genetically modified to express one or more proteins that support, activate, or provide a desired function to the transfected cells. Such additional genetic modification may be separately performed, that is, before transfection with the nucleic acid encoding CXADR, or contemporaneously, that is, together with the transfection with the nucleic acid encoding CXADR (e.g., from the same recombinant nucleic acid or from a second recombinant nucleic acid).

For example, the NK or other host cells may express at least a portion of IL2RA, optionally together with one or more of IL2RB and IL2RG to provide an extra avenue for NK cell activation and to so enhance a more robust immune response. Genetically engineered NK cells will most preferably be activated NK cells, high-affinity NK cells, or target activated NK cells. Preferred IL2RA include full length or high-affinity variants of IL2RA. In addition, it is contemplated that the genetically engineered NK cells may also express one or more cytokines, and especially IL-12. Thus, it should be appreciated that the so prepared NK cells may outcompete the hosts T-cells for IL2. Moreover, contemplated NK or other host cells may also express IL-15 or a IL-15 superagonist (e.g., ALT-803) to so provide increased activation. Finally, where desired, the NK or other host cells may express one or more immune checkpoint inhibitors to further enhance or stimulate the host immune response.

In yet another example, the inventors contemplate transfection of genetically engineered NK or other host cells to express one or more co-stimulatory molecules to so enhance an immune response. Once more, the genetically engineered NK cells will most preferably be activated NK cells, high-affinity NK cells, or target activated NK cells. Preferred co-stimulatory molecules can be B7.1 (CD80), ICAM-1 (CD54), ICOS-L, and/or LFA-3 (CD58). In another example, preferred co-stimulatory molecules can be 4-1BBL, CD30L, CD40, CD40L, CD48, CD70, CD112, CD155, GITRL, OX40L, and/or TL1A, optionally in combination with any one of B7.1 (CD80), ICAM-1 (CD54), ICOS-L, and/or LFA-3 (CD58).

Where desired, modified NK cells may also present at least a portion of CXCL12, more preferably a full length CXCL12, and/or that the NK cells are genetically modified to reduce or even entirely silence expression of the CXCR4. By presentation of at least a portion of CXCL12 on the surface of the NK cells and/or removal of the CXCR4, it is believed that the so modified cells will be less subject to recognition and allograft rejection by the host and will have a reduced propensity to aggregate, while still retaining killing activity via NK cell-specific pathways.

Moreover, it should be recognized that while immune competent cells are generally preferred for expression of the CXADR, numerous non-immune competent cells are also deemed suitable and especially include established cells lines suitable for recombinant protein production. Thus, various mammalian and insect cell lines are particularly contemplated, including CHO cells, HEK-293 cells, mouse myeloma lymphoblastoid cells, BHK cells, Sf9, CV-1, COS-1 cells, etc.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of modifying an immune competent cell, comprising:
introducing into the immune competent cell a recombinant nucleic acid encoding CXADR to produce a modified immune competent cell, wherein the immune competent cell is an NK cell, a T-cell, a B-cell, a macrophage, or a dendritic cell;
cultivating the modified immune competent cell to a desired quantity in a first medium under conditions to express the CXADR; and
replacing the first medium with a second medium suitable for administration of the modified immune competent cell to a mammal.

2-4. (canceled)

5. The method of claim 1, wherein the recombinant nucleic acid encoding CXADR is under the control of a hypoxia inducible promoter.

6. The method of claim 1 further comprising a step of infecting the modified immune competent cell with a recombinant adenovirus.

7. The method of claim 6 wherein the recombinant adenovirus has an E2b deletion and includes a recombinant nucleic acid that encodes at least one of a neoepitope, a co-stimulatory molecule, a cytokine, and a checkpoint inhibitor.

8. The method of claim 6 further comprising a step of administering the modified immune competent cell to a patient, and wherein the step of infecting is performed in vivo after administration of the modified immune competent cell to the patient, or further comprising a step of administering the modified immune competent cell to a patient, wherein the step of infecting is performed in vitro before administration of the modified immune competent cell to the patient.

9-25. (canceled)

26. A method of conditioning a patient for immunotherapy of a cancer, comprising a step of administering to the patient an immune competent cell that is genetically modified to express CXADR.

27. The method of claim 26 wherein the immune competent cell is an NK cell, a T-cell, a B-cell, a macrophage, or a dendritic cell.

28. The method of claim 26 further comprising a step of infecting the immune competent cell with a recombinant adenovirus that comprises a nucleic acid that encodes at least one of a neoepitope, a co-stimulatory molecule, a cytokine, and a checkpoint inhibitor.

29. The method of claim 26 further comprising a step of administering to the patient a recombinant adenovirus that comprises a nucleic acid that encodes at least one of a neoepitope, a co-stimulatory molecule, a cytokine, and a checkpoint inhibitor.

30. The method of claim 28 or claim 29 wherein the recombinant adenovirus has a deleted or non-functional E2b gene.

31. A method of treating a patient diagnosed with cancer, comprising:
administering to the patient a genetically modified immune competent cell that comprises a recombinant nucleic acid that encodes CXADR and in which the nucleic acid that encodes CXADR is operably coupled to a regulatory sequence for expression of the CXADR in the immune competent host cell;
administering to the patient a recombinant adenovirus that comprises a nucleic acid that encodes at least one of a neoepitope, a co-stimulatory molecule, a cytokine, and a checkpoint inhibitor; and
wherein the recombinant adenovirus is administered upon expression of the CXADR in the genetically modified immune competent cell in the patient.

32. The method of claim 31 wherein immune competent cell is an NK cell, a T-cell, a B-cell, a macrophage, or a dendritic cell.

33. The method of claim 31 wherein the recombinant adenovirus has a deleted or non-functional E2b gene.

34. The method of claim 31 wherein the genetically modified immune competent cell is an autologous cell of the patient.

35. The method of claim 31 wherein the genetically modified immune competent cell is a genetically modified immune competent cell according to any one of claims 12-25.

36. A method of treating a patient diagnosed with cancer, comprising:
infecting a genetically modified immune competent cell with a recombinant adenovirus;
wherein the genetically modified immune competent cell comprises a recombinant nucleic acid that encodes CXADR and in which the nucleic acid that encodes CXADR is operably coupled to a regulatory sequence for expression of the CXADR in the immune competent host cell;
wherein the recombinant adenovirus comprises a nucleic acid that encodes at least one of a neoepitope, a co-stimulatory molecule, a cytokine, and a checkpoint inhibitor; and
administering to the patient the infected immune competent cell.

37. The method of claim 36 wherein immune competent cell is an NK cell, a T-cell, a B-cell, a macrophage, or a dendritic cell.

38. The method of claim 36 wherein the recombinant adenovirus has a deleted or non-functional E2b gene.

39. The method of claim 36 wherein the genetically modified immune competent cell is an autologous cell of the patient.

40. The method of claim 36 wherein the neoepitope is a cancer and patient-specific neoepitope.

41-42. (canceled)


SEQUENCE LISTING (1 sequence)

SEQ ID NO: 1 - DNA, 1112 bp, Homo sapiens
  gene (7)..(1110): CXADR isoform 1
aagcttatggcgctcctgctgtgcttcgtgctcctgtgcggagtagtggatttcgccagaagtttgagtatcactactcctgaagagatgattgaaaaagccaaaggggaaactgcctatctgccatgcaaatttacgcttagtcccgaagaccagggaccgctggacatcgagtggctgatatcaccagctgataatcagaaggtggatcaagtgattattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttgttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcagaaatgacttcatctgttatatctgtaaaaaatgcctcttctgagtactctgggacatacagctgtacagtcagaaacagagtgggctctgatcagtgcctgttgcgtctaaacgttgtccctccttcaaataaagctggactaattgcaggagccattataggaactttgcttgctctagcgctcattggtcttatcatcttttgctgtcgtaaaaagcgcagagaagaaaaatatgaaaaggaagttcatcacgatatcagggaagatgtgccacctccaaagagccgtacgtccactgccagaagctacatcggcagtaatcattcatccctggggtccatgtctccttccaacatggaaggatattccaagactcagtataaccaagtaccaagtgaagactttgaacgcactcctcagagtccgactctcccacctgctaaggtagctgcccctaatctaagtcgaatgggtgcgattcctgtgatgattccagcacagagcaaggatgggtctatagtataggcggccgc